(19) United States
(12) Patent Application Publication
(10) Pub. No.: US 2024/0168114 A1
(43) Pub. Date: May 23, 2024

(54) LEARNING APPARATUS AND ESTIMATION APPARATUS

(71) Applicant: JVCKENWOOD Corporation, Yokohama-shi (JP)

(72) Inventor: Hideki Takehara, Yokohama-shi (JP)

(21) Appl. No.: 18/424,910

(22) Filed: Jan. 29, 2024

Related U.S. Application Data
(63) Continuation of application No. PCT/JP2022/029383, filed on Jul. 29, 2022.

(30) Foreign Application Priority Data
Jul. 30, 2021 (JP) 2021-125652

(51) Int. Cl. G01R 33/48; A61B 5/00

(52) U.S. Cl. CPC G01R 33/4806; A61B 5/0042; A61B 5/0075; A61B 5/4064; G01R 33/4808

(57) ABSTRACT

A learning apparatus includes: an acquisition unit configured to acquire a functional image that is based on a measurement result obtained by measuring a brain activity state of a subject by functional near-infrared spectroscopy; and an associated data generation unit configured to generate associated data in which the acquired functional image and brain activity information indicating states of Wernicke area and Broca area are associated with each other, the brain activity information being information indicating the brain activity state of the subject at a time of measurement by the functional near-infrared spectroscopy and being based on information different from the measurement result obtained by the functional near-infrared spectroscopy.

### CROSS-REFERENCE TO RELATED APPLICATIONS

This application is a Continuation of PCT International Application No. PCT/JP2022/029383 filed on Jul. 29, 2022 which claims the benefit of priority from Japanese Patent Application No. 2021-125652 filed on Jul. 30, 2021, the entire contents of both of which are incorporated herein by reference.

### BACKGROUND OF THE INVENTION

The present application relates to a learning apparatus and an estimation apparatus.

As a method of measuring a brain activity state of a subject, functional near-infrared spectroscopy is known (for example, see Japanese Laid-open Patent Publication No. 2009-136434). The functional near-infrared spectroscopy does not need a large-scale facility as compared to other measurement methods such as functional magnetic resonance imaging, therefore has a high degree of freedom for measurement and can be realized at low cost.

However, the functional near-infrared spectroscopy has a problem in that the functional near-infrared spectroscopy has low spatial resolution and is easily affected by noise such as a scalp blood flow. Therefore, there is a need to improve accuracy of a measurement result.

A learning apparatus and an estimation apparatus are disclosed.

According to one aspect of the present application, there is provided a learning apparatus comprising: an acquisition unit configured to acquire a functional image that is based on a measurement result obtained by measuring a brain activity state of a subject by functional near-infrared spectroscopy; and an associated data generation unit configured to generate associated data in which the acquired functional image and brain activity information indicating states of Wernicke area and Broca area are associated with each other, the brain activity information being information indicating the brain activity state of the subject at a time of measurement by the functional near-infrared spectroscopy and being based on information different from the measurement result obtained by the functional near-infrared spectroscopy.

According to one aspect of the present application, there is provided an estimation apparatus comprising: an acquisition unit configured to acquire a functional image that is based on a measurement result obtained by measuring a brain activity state of a subject by functional near-infrared spectroscopy; and an estimation unit configured to estimate a brain activity state of the subject based on the functional image acquired by the acquisition unit and a trained model that is generated in advance by machine learning with respect to a correlation between the functional image and brain activity information indicating states of Wernicke area and Broca area of the subject.

The above and other objects, features, advantages and technical and industrial significance of this application will be better understood by reading the following detailed description of presently preferred embodiments of the application, when considered in connection with the accompanying drawings.

### BRIEF DESCRIPTION OF THE DRAWINGS

FIG. 1 is a functional block diagram illustrating an example of a brain function learning estimation system according to the present embodiment;

FIG. 2 is a diagram illustrating an example of a neural network;

FIG. 3 is a diagram schematically illustrating an example of a learning method;

FIG. 4 is a diagram schematically illustrating an example of an estimation method;

FIG. 5 is a flowchart illustrating an example of operation of a learning apparatus: and

FIG. 6 is a flowchart illustrating an example of operation of an estimation apparatus.

### DETAILED DESCRIPTION OF THE INVENTION

Embodiments of a learning apparatus and an estimation apparatus according to the present application will be described below based on the drawings. The present application is not limited by the embodiments below. Further, components in the embodiments described below include one that can easily be replaced by a person skilled in the art and one that is practically identical.

### First Embodiment

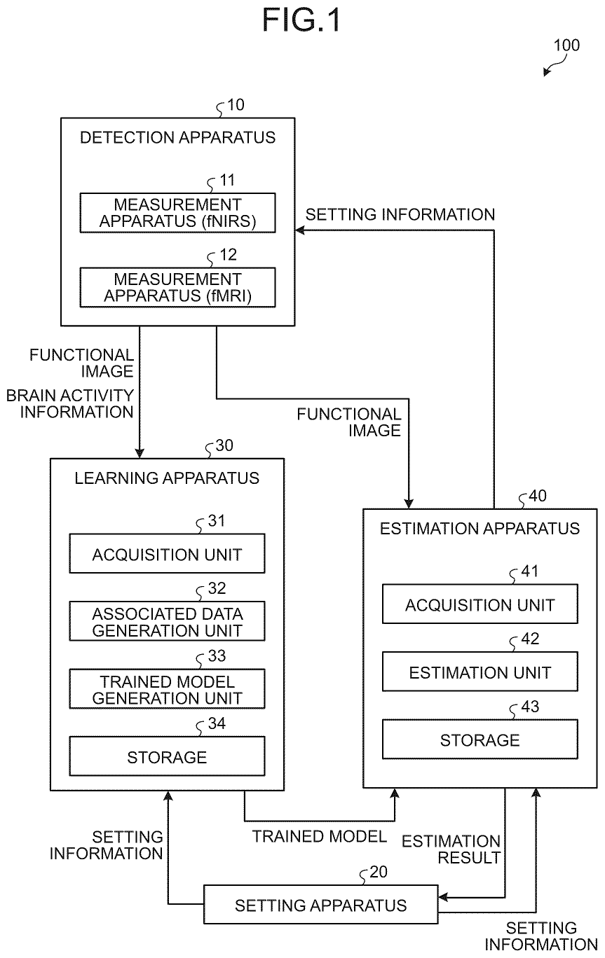

FIG. 1 is a functional block diagram illustrating an example of a brain function learning estimation system 100 according to the present embodiment. The brain function learning estimation system 100 illustrated in FIG. 1 includes a detection apparatus 10, a setting apparatus 20, a learning apparatus 30, and an estimation apparatus 40.

The detection apparatus 10 detects brain activity information that indicates a brain activity state of a subject. The detection apparatus 10 includes, for example, a measurement apparatus 11 that measures a cerebral blood flow based on principle of functional Near-Infrared Spectroscopy (fNIRS) and a measurement apparatus 12 that is not limited to a measurement apparatus based on principle of fNIRS and that performs measurement based on principle of functional Magnetic Resonance Imaging (fMRI) for measuring an amount of brain activity as the brain activity information for example.

The measurement apparatus 11 generates a functional image that represents the brain activity state of the subject from a result of measurement that is based on the principle of fNIRS. Examples of the functional image include an image that represents a distribution of at least one of an oxygenated hemoglobin concentration, a deoxygenated hemoglobin concentration, and a total hemoglobin concentration in a predetermined site of the brain of the subject. Meanwhile, it may be possible to generate, as the functional image, an image that represents a distribution of each of the oxygenated hemoglobin concentration, the deoxygenated hemoglobin concentration, and the total hemoglobin concentration. Further, it may be possible to generate only the functional image of the deoxygenated hemoglobin concentration that is presumed to be most correlated with the brain activity state. Furthermore, it may be possible to integrate the functional images of the oxygenated hemoglobin concentration, the deoxygenated hemoglobin concentration, and the total hemoglobin concentration into a single functional image while adopting each of the functional images as a single channel. For example, in a case of an RGB image, each of R, G, and B corresponds to a channel. With this configuration, it is possible to learn, by machine learning, a spatial correlation among the functional images of the oxygenated hemoglobin concentration, the deoxygenated hemoglobin concentration, and the total hemoglobin concentration.

The functional image is an image that represents a cross section of the brain of the subject. The functional image can be represent by a cross section of the brain of the subject that is cut along at least a single plane such as a transverse plane, a sagittal plane, and a coronal plane, for example. A type of the cross section may be set by the setting apparatus 20 for example, and setting information set by the setting apparatus 20 is transferred to the detection apparatus 10 via the estimation apparatus 40.

As the predetermined site of the brain as a target for the functional image, for example, a language center such as Wernicke area and Broca area may be adopted. When Wernicke area and Broca area are adopted as the predetermined sites, it is preferable to adopt, as the functional image, at least one of the transverse plane and the sagittal plane that is suitable for measuring the both areas, for example. Meanwhile, the predetermined site is not limited to those as described above, but may be any other site as long as the site can be measured by fNIRS. In fNIRS, it is possible to preferably measure a site on a surface side such as cerebrum (neocortex) in the brain of the subject, for example. Therefore, at least a part of the cerebrum of the subject may be adopted as the predetermined site, for example.

The measurement apparatus 11 that measures the cerebral blood flow based on principle of fNIRS includes, for example, multiple optical channels. The optical fiber channels include emission optical fibers for emitting near-infrared light from a light source to a head of the subject and reception optical fibers for receiving near-infrared light that is reflected or scattered inside the head of the subject. The emission optical fibers and the reception optical fibers are arranged at positions at which brain activity information on Wernicke area and Broca area of the subject is measurable.

The measurement apparatus 11 is able to generate a functional image by, for example, a diffuse optical tomography method or the like based on the type of the cross section as described above (the transverse plane, the sagittal plane, or the coronal plane) and the predetermined site (Wernicke area, Broca area, or the like).

The measurement apparatus 12 generates a reference image that represents the brain activity state of the subject from a result of the measurement that is based on principle of fMRI. The reference image is, for example, an image that represents a change of the oxygenated hemoglobin concentration, the deoxygenated hemoglobin concentration, or the total hemoglobin concentration in the predetermined site with respect to the image of the brain of the subject, and may be an image in which the respective images are overlapped. Further, it is desirable to adopt, as the reference image, an image that is correlated with at least the functional image that is generated by the measurement apparatus 11. For example, if the functional image generated by the measurement apparatus 11 is the functional image of the deoxygenated hemoglobin concentration, the measurement apparatus 12 generates, as the reference image, an image that represents a change of the deoxygenated hemoglobin concentration. fMRI has advantages that fMRI has high spatial resolution and is not easily affected by noise as compared to fNIRS. Therefore, the reference image obtained by fMRI makes it possible to determine the brain activity state of the subject with high accuracy as compared to the functional image is obtained by fNIRS.

The setting apparatus 20 inputs setting information on the brain function learning estimation system 100. As the setting apparatus 20, an input apparatus such as a keyboard or a mouse may be used, or a smartphone, a table, or the like that includes a touch panel may be used.

The learning apparatus 30 learns a detection result of the detection apparatus 10. The learning apparatus 30 includes, for example, a processing apparatus such as a Central Processing Unit (CPU), and a storage apparatus such as a Random Access Memory (RAM) or a Read Only Memory (ROM). The learning apparatus 30 includes an acquisition unit 31, an associated data generation unit 32, a trained model generation unit 33, and a storage 34.

The acquisition unit 31 acquires the functional image based on a measurement result obtained by measuring the brain activity state of the subject by fNIRS. In the present embodiment, the acquisition unit 31 acquires the functional image based on the measurement result of the measurement apparatus 11. Further, in the present embodiment, the acquisition unit 31 acquires, for example, the functional image that represents Wernicke area and Broca area of the brain of the subject.

The associated data generation unit 32 generates associated data in which the functional image acquired by the acquisition unit 31 and brain activity information corresponding to the functional image are associated with each other. The brain activity information is information that indicates the brain activity state of the subject at the time of measurement by fNIRS and that is based on information different from the measurement result obtained by fNIRS. Examples of the brain activity information as described above include information that indicates activity states of Wernicke area and Broca area of the brain of the subject. In the present embodiment, the brain activity information is information that is based on the measurement result obtained by measuring the brain of the subject by fMRI. In other words, the brain activity information indicates an activity state that can be determined based on the reference image generated by the measurement apparatus 12, for example. The activity state may be determined manually based on the reference image, for example. Meanwhile, it may be possible to automatically determine the activity state by an image processing apparatus or the like.

The activity state of each of Wernicke area and Broca area may be set in stages. For example, the activity state of each of Wernicke area and Broca area may be set in two stages such as “high activity” and “low activity”. Meanwhile, while it is explained that the activity state of each of the areas can be set in stages, the stages may be set in accordance with characteristics of each of the areas. For example, based on a predetermined threshold W1, the activity state of Wernicke area is classified as high activity if the activity state is equal to or larger than the predetermined threshold W1 and is classified as low activity if the activity state is smaller than the predetermined threshold W1. Further, based on a predetermined threshold B1, the activity state of Broca area is classified as high activity if the activity state is equal to or larger than the predetermined threshold B1 and is classified as low activity if the activity state is smaller than the predetermined threshold B1. One example of setting of the predetermined threshold for Wernicke area will be described below. The brain activity information in a predetermined time period is measured while giving or not giving a stimulus that responds to the five senses such as an auditory stimulus or a visual stimulus, a large number of reference images are generated in the predetermined time period, and a lowest value and a highest value of pixel values (pixel values converted from at least one of the oxygenated hemoglobin concentration, the deoxygenated hemoglobin concentration, and the total hemoglobin concentration) of a region corresponding to Wernicke area in the large number of the reference images in the predetermined time period are acquired. Further, the predetermined threshold may be set to 50% of the highest value as described above, the predetermined threshold may be set to 150% of the lowest value, or the predetermined threshold may be set to an intermediate value between the highest value and the lowest value. In this manner, the predetermined threshold is set based on the pixel values of the reference images. The threshold for Broca area may be set in the same manner as described above. Meanwhile, the activity state of each of the areas may be set in three or more stages.

Specifically, the activity states of Wernicke area and Broca area may be classified into the following four stages.
(1) Wernicke area is in a low activity state and Broca area is in a low activity state (a low activity state in both areas)
(2) Wernicke area is in a low activity state and Broca area is in a high activity state (a Broca area dominant state)
(3) Wernicke area is in a high activity state and Broca area is in a low activity state (a Wernicke area dominant state)
(4) Wernicke area is in a high activity state and Broca area is in a high activity state (a high activity state in both-areas).

Therefore, as the brain activity information, four states such as a low activity state in both areas, a Broca area dominant state, a Wernicke area dominant state, and a high activity state in both areas, may be set. In this example, the activity state of each of Wernicke area and Broca area is set in the same number of stages, but may be set in a different number of stages, such that, for example, Wernicke area is set in two stages and Broca area is set in four stages.

In the present embodiment, the associated data generation unit 32 generates associated data in which the functional image based on the measurement results by simultaneously measuring the brain of the same subject by the measurement apparatus 11 and the measurement apparatus 12 and the brain activity information are associated with each other. Specifically, in the associated data, the brain activity information that is determined based on the measurement result by fMRI is associated with the functional image that is based on the measurement result by fNIRS. As described above, the reference image obtained by fMRI makes it possible to determine the brain activity state of the subject with high accuracy as compared to the functional image obtained by fNIRS. Therefore, the possibility that highly accurate information is associated with the functional image increases as compared to information that is obtained by determining the brain activity state of the subject based on the functional image obtained by fNIRS. The associated data generation unit 32 stores the generated associated data in the storage 34.

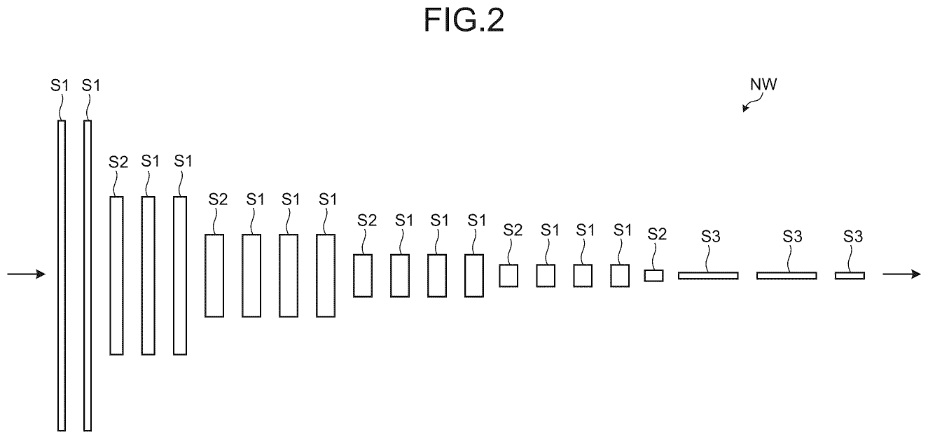

In the present embodiment, the trained model generation unit 33 generates a trained model by using a neural network (convolutional neural network) that is represented by VGG16, for example. FIG. 2 is a diagram illustrating an example of the neural network. As illustrated in FIG. 2, a neural network NW includes 13 convolutional layers S1, five pooling layers S2, and three fully-connected layers S3. In the neural network, input information is sequentially subjected to processes in the convolutional layers S1 and the pooling layers S2, and processing results are connected in and output from the fully-connected layers S3.

The trained model generation unit 33 inputs the associated data generated by the associated data generation unit 32 to the neural network NW, and learns, by machine learning such as deep learning, a correlation between the functional image and the brain activity information that are associated with each other in the associated data. Specifically, the trained model generation unit 33 optimizes the neural network NW by training and generates a trained model. For example, in supervised learning, the brain activity information is handled as ground truth data, and, training is performed so as to solve a problem with classification into four states since the brain activity information indicates four states in this example. Meanwhile, in the present embodiment, the example is described in which the trained model is generated by using the convolutional neural network represented by VGG16, but embodiments are not limited to this example, and it may be possible to generate a trained model by using a different kind of neural network.

The storage 34 stores therein various kinds of information. The storage 34 includes, for example, a storage such as a hard disk drive or a solid state drive. Meanwhile, it may be possible to use, as the storage 34, an external storage medium such as a removable disk. The storage 34 stores therein the associated data that is generated by the associated data generation unit 32 and the trained model that is generated by the trained model generation unit 33.

The storage 34 stores therein a training program that causes a computer to perform a process of acquiring the functional image that is based on a measurement result by measuring the brain activity state of the subject by fNIRS and a process of generating the associated data in which the acquired functional image and the brain activity information that is information indicating the brain activity state of the subject at the time of measurement by fNIRS and that is based on information different from fNIRS are associated with each other. The storage 34 stores therein a training program that further causes the computer to perform a process of generating a trained model by performing machine learning on a correlation between the functional image and the brain activity information based on the generated associated data.

The estimation apparatus 40 estimates the brain activity state of the subject. The estimation apparatus 40 includes, for example, a processing apparatus such as a CPU, and a storage such as a RAM or a ROM. The estimation apparatus 40 includes an acquisition unit 41, an estimation unit 42, and a storage 43.

The acquisition unit 41 acquires the functional image that is based on the measurement result by measuring the brain activity state of the subject by fNIRS. The acquisition unit 41 acquires the functional image generated by the measurement apparatus 11.

The estimation unit 42 estimates the brain activity state of the subject based on the functional image acquired by the acquisition unit 41 and the trained model generated by the learning apparatus 30. The estimation unit 42 inputs the functional image acquired by the acquisition unit 41 to the trained model. In this case, the trained model outputs the brain activity information corresponding to the input functional image based on a learning result of the correlation between the functional image and the brain activity information. The estimation unit 42 estimates that the brain activity information as an output result indicates the brain activity state of the subject. The estimation unit 42 outputs an estimation result. The output estimation result is transmitted to the setting apparatus 20 and displayed by the setting apparatus 20, for example. Further, the estimation unit 42 may store the estimation result in the storage 43.

The storage 43 stores therein various kinds of information. The storage 43 includes, for example, a storage such as a hard disk drive or a solid state drive. Meanwhile, it may be possible to use, as the storage 43, an external storage medium such as a removable disk. The storage 43 is able to store therein the trained model that is generated by the learning apparatus 30.

The storage unit 43 stores a program that causes a computer to perform a process of acquiring the functional image that is based on a measurement result by measuring the brain activity state of the subject by fNIRS and a process of estimating the brain activity state of the subject based on the acquired functional image and the trained model generated in advance by machine learning with respect to the correlation between the functional image and the brain activity information that indicates the brain activity state of the subject at the time of measurement of the functional image.

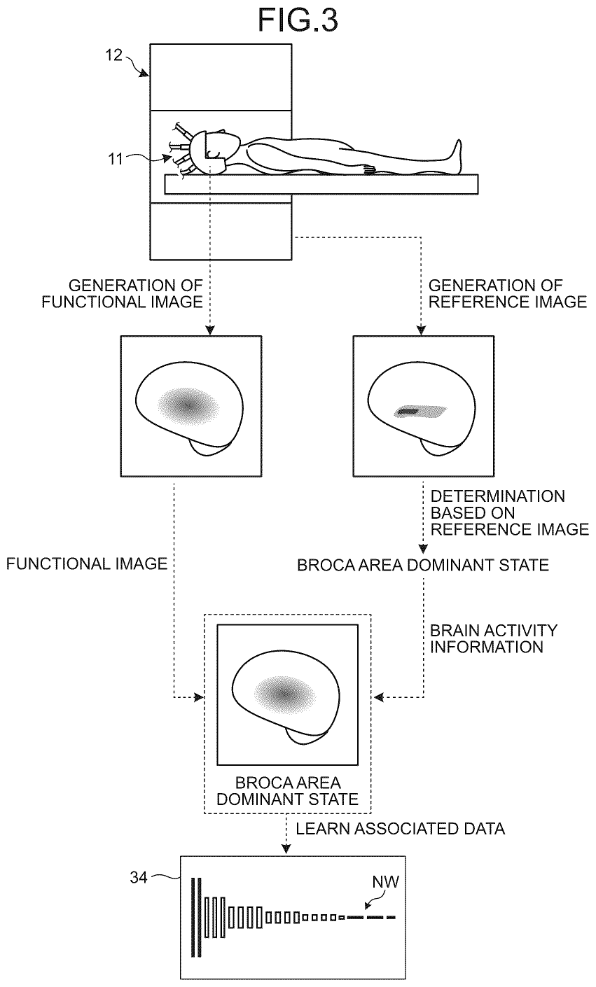

A learning method and an estimation method using the brain function learning estimation system 100 configured as described above will be described below. FIG. 3 is a diagram schematically illustrating a flow of the learning method. In the learning method according to the present embodiment, the functional image and the brain activity information that serve as materials are first generated. A subject is measured simultaneously by the measurement apparatus 11 and the measurement apparatus 12. The measurement apparatus 11 and the measurement apparatus 12 generate the functional image and the reference image based on measurement results.

A determiner determines the brain activity information on the subject by viewing the reference image. The reference image is highly accurate as compared to the functional image, so that the possibility that a highly accurate determination result is obtained increases as compared to determination of the brain activity information of the subject with reference to the functional image. The determiner determines the brain activity state of the subject while viewing the reference image, and inputs the brain activity information as a determination result to the setting apparatus 20. In this example, for example, a determination result indicating a Broca area dominant state is input. The input brain activity information is stored in, for example, a memory or the like of the measurement apparatus 12. Meanwhile, the functional image and the brain activity information that are based on the results of the measurement performed simultaneously may include tag information, such as measurement times, so as to enable association therebwtween. By performing the measurement and the determination as described above multiple number of times, multiple combinations of the functional images and the brain activity information are accumulated.

In this state, by using the setting apparatus 20 to cause the learning apparatus 30 to start learning, a learning process is started. The acquisition unit 31 acquires the functional image that is based on the measurement result of the measurement apparatus 11. The associated data generation unit 32 acquires the brain activity information from the measurement apparatus 12. The associated data generation unit 32 generates the associated data in which the acquired functional image and the brain activity information are associated with each other based on tag information or the like. The associated data generation unit 32 stores the generated associated data in the storage 34.

The trained model generation unit 33 performs machine learning (deep learning) on the correlation between the functional image and the brain activity information based on the generated associated data, and therefore optimize the neural network NW and generate a trained model. The trained model generation unit 33 stores the generated trained model in the storage 34.

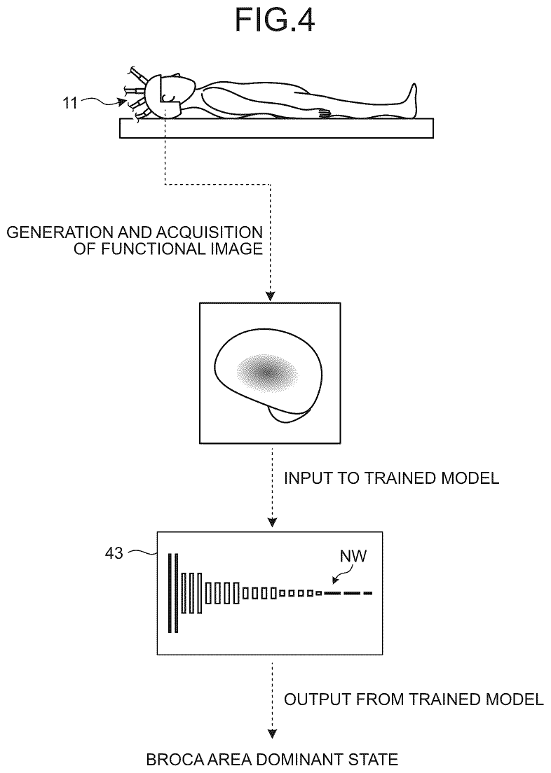

FIG. 4 is a diagram schematically illustrating a flow of the estimation method. In the estimation method according to the present embodiment, the subject is measured by the measurement apparatus 11. The measurement apparatus 11 generates the functional image based on the measurement result. In this state, by the setting apparatus 20 to cause the estimation apparatus 40 to start estimation, an estimation process is started. The estimation apparatus 40 acquires, in advance, the trained model that is stored in the storage 34 of the learning apparatus 30 and stores the acquired trained model in the storage 43.

The acquisition unit 41 acquires the functional image that is based on the measurement result of the measurement apparatus 11. The estimation unit 42 inputs the functional image to the neural network NW that is the trained model stored in the storage unit 43, acquires the brain activity information that is an output result, and estimates that the acquired brain activity information indicates the brain activity state of the subject. In the example illustrated in FIG. 4, the neural network NW outputs the brain activity information indicating “a Broca area dominant state”. The estimation unit 42 estimates that the brain activity state of the subject is “a Broca area dominant state” based on the output result from the neural network NW. The estimation unit 42 outputs the estimation result. The estimation result output from the estimation unit 42 may be displayed by, for example, an external apparatus such as the setting apparatus 20.

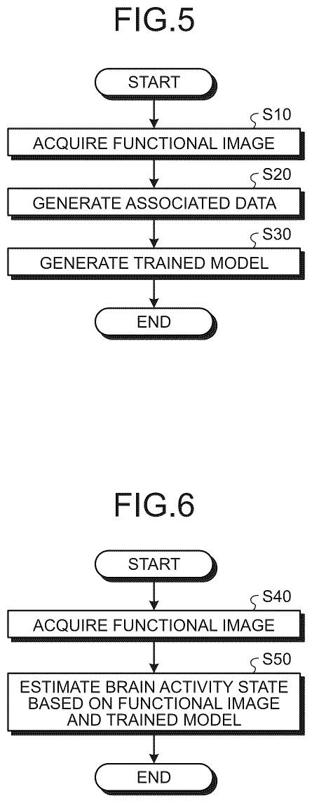

FIG. 5 is a flowchart illustrating an example of operation of the learning apparatus 30. As illustrated in FIG. 5, in the learning apparatus 30, the acquisition unit 31 acquires the functional image that is based on the result of the measurement of the brain activity state of the subject by fNIRS (Step S10). Subsequently, the associated data generation unit 32 generates the associated data in which the acquired functional image and the brain activity information that is information indicating the brain activity state of the subject at the time of measurement by fNIRS and that is based on information different from the measurement result by fNIRS are associated with each other (Step S20). Then, the trained model generation unit 33 generates the trained model by performing machine learning on the correlation between the functional image and the brain activity information based on the associated data generated by the associated data generation unit 32 (Step S30).

FIG. 6 is a flowchart illustrating an example of operation of the estimation apparatus 40. As illustrated in FIG. 6, in the estimation apparatus 40, the acquisition unit 41 acquires the functional image that is based on a measurement result by measuring the brain activity state of the subject by fNIRS (Step S40). Subsequently, the estimation unit 42 estimates the brain activity state of the subject based on the trained model that is generated in advance with respect to the correlation between the functional image and the brain activity information and based on the functional image that is acquired by the acquisition unit 41 (Step S50).

As described above, the learning apparatus 30 according to the present embodiment includes the acquisition unit 31 that acquires the functional image that is based on a measurement result by measuring the brain activity state of the subject by fNIRS, and the associated data generation unit 32 that generates the associated data in which the acquired functional image and the brain activity information that is information indicating the brain activity state of the subject at the time of measurement by fNIRS and that is based on information different from the measurement result by fNIRS are associated with each other.

With this configuration, it is possible to associate the functional image based on the measurement result by fNIRS and the brain activity information based on information different from the measurement result by fNIRS. Therefore, it is possible to associate the functional image and the brain activity information with high accuracy as compared to a case in which the brain activity information is determined from the functional image that is based on the measurement result by fNIRS. Consequently, it is possible to contribute to highly accurate measurement of the brain activity state of the subject.

The learning apparatus 30 according to the present embodiment further includes the trained model generation unit 33 that generates the trained model by performing machine learning on the correlation between the functional image and the brain activity information based on the associated data that is generated by the associated data generation unit 32. With this configuration, the trained model is generated by using the associated data in which the functional image and the brain activity information are associated with each other with high accuracy, so that it is possible to generate the trained model for the correlation between the functional image and the brain activity information with high accuracy. Therefore, it is possible to contribute to highly accurate measurement of the brain activity state of the subject.

In the learning apparatus 30 according to the present embodiment, the information different from the measurement result by fNIRS is information that is based on a measurement result by measuring the brain of the subject by fMRI at a time corresponding to the measurement by fNIRS. fMRI has advantages that fMRI has high spatial resolution and is not easily affected by noise as compared to fNIRS. Therefore, by the reference image that is obtained by fMRI, it is possible to determine the brain activity state of the subject with high accuracy as compared to the case by the functional image that is obtained by fNIRS. Consequently, it is possible to associate the functional image and the brain activity information with high accuracy as compared to a case in which the brain activity information is determined based on the functional image from the measurement result by fNIRS.

In the learning apparatus 30 according to the present embodiment, the functional image is an image that represents a cerebrum of the subject. With this configuration, the image of the cerebrum that can be measured by fNIRS in a preferable manner is used, so that it is possible to perform association and learning with high accuracy.

The estimation apparatus 40 according to the present embodiment includes the acquisition unit 41 that acquires the functional image that is based on a measurement result by measuring the brain activity state of the subject by fNIRS, and the estimation unit 42 that estimates the brain activity state of the subject based on the functional image that is acquired by the acquisition unit 41 and the trained model that is generated in advance by machine learning with respect to the correlation between the functional image and the brain activity information that indicates the brain activity state of the subject at the time of measurement of the functional image.

With this configuration, it is possible to estimate the brain activity state of the subject based on the functional image that is based on the measurement result by fNIRS and the trained model that is generated in advance by the machine learning, so that it is possible to estimate the brain activity state of the subject with high accuracy as compared to a case in which the estimation is performed manually from the functional image that is based on the measurement result by fNIRS.

Meanwhile, it is explained that the activity state of each of Wernicke area and Broca area can be set in stages, but may be set based on characteristics of the optical fiber channels of fNIRS. Specifically, the activity states of Wernicke area and Broca area are set in stages based on characteristics of the optical fiber channels of fNIRS. An example in which the stages of the activity states of Wernicke area and Broca area are changed based on the characteristics of the optical fiber channels of fNIRS will be described below as a modification.

The activity states of Wernicke area and Broca area are set in stages in accordance with the number of the optical fiber channels of fNIRS. For example, the number of stages indicating the activity states of Wernicke area is set in proportion to the number of the reception optical fibers that are installed to measure the brain activity information on Wernicke area. Further, the number of stages indicating the activity states of Broca area is set in proportion to the number of the reception optical fibers that are installed to measure the brain activity information of Broca area. In this example, the number of the optical fiber channels is adopted as the number of the reception optical fibers, but may be adopted as a total number of the reception optical fibers and the emission optical fibers. With this configuration, it is possible to appropriately learn and measure the brain activity information in accordance with the number of the mounted optical fiber channels of fNIRS.

The activity states of Wernicke area and Broca area are set in accordance with an output amount of the emission optical fibers or sensitivity of the reception optical fibers among the optical fiber channels of fNIRS. For example, the stages that represent the activity states of Wernicke area are set in proportion to the sensitivity of the reception optical fibers that is installed to measure the brain activity information on Wernicke area. Further, the stages that represent the activity states of Broca area are set in proportion to the sensitivity of the reception optical fibers that is installed to measure the brain activity information on Broca area. In this example, the stages may be set in proportion to the sensitivity of the reception optical fibers, but it may be possible to adopt the output amount of the emission optical fibers or it may be possible to take into account both of the sensitivity of the reception optical fibers and the output amount of the emission optical fibers. Further, the stages may be set in proportion to a diameter size of the reception optical fiber or a diameter size of the emission optical fiber for acquiring the sensitivity. With this configuration, it is possible to appropriately learn and measure the brain activity information in accordance with the output amount or the sensitivity of the mounted optical fiber channels of fNIRS.

The technical scope of the present application is not limited to the embodiments as described above, and appropriate modifications may be made without departing from the scope of the present application. For example, in the embodiments as described above, the examples have been described in which the learning apparatus 30 and the estimation apparatus 40 constitute a part of the brain function learning estimation system 100, but embodiments are not limited thereto. A configuration in which the learning apparatus 30 and the estimation apparatus 40 are arranged as independent apparatuses or systems may be applicable.

Furthermore, in the embodiment as described above the configuration in which the learning apparatus 30 and the estimation apparatus 40 are provided as separate apparatuses is explained as an example, but embodiments are not limited thereto. For example, a configuration in which a processing apparatus that have functions of the learning apparatus 30 and functions of the estimation apparatus 40 may be applicable.

The learning apparatus and the estimation apparatus according to the present application can be used in a processing apparatus such as a computer, for example.

According to the present application, it is possible to provide a learning apparatus and an estimation apparatus for measuring a brain activity state of a subject with high accuracy.

Although the application has been described with respect to specific embodiments for a complete and clear application, the appended claims are not to be thus limited but are to be construed as embodying all modifications and alternative constructions that may occur to one skilled in the art that fairly fall within the basic teaching herein set forth.

What is claimed is:

1. A learning apparatus comprising:
an acquisition unit configured to acquire a functional image that is based on a measurement result obtained by measuring a brain activity state of a subject by functional near-infrared spectroscopy; and
an associated data generation unit configured to generate associated data in which the acquired functional image and brain activity information indicating states of Wernicke area and Broca area are associated with each other, the brain activity information being information indicating the brain activity state of the subject at a time of measurement by the functional near-infrared spectroscopy and being based on information different from the measurement result obtained by the functional near-infrared spectroscopy.

2. The learning apparatus according to claim 1, wherein the brain activity information indicates a low activity state in Wernicke area and Broca area, a Broca area dominant state, a Wernicke area dominant state, and a high activity state in Wernicke area and Broca area.

3. The learning apparatus according to claim 1, further comprising:
a trained model generation unit configured to generate a trained model by performing machine learning on a correlation between the functional image and the brain activity information based on the associated data generated by the associated data generation unit.

4. The learning apparatus according to claim 1, wherein the information different from the measurement result obtained by the functional near-infrared spectroscopy is information that is based on a measurement result obtained by measuring the brain of the subject by functional magnetic resonance imaging at a time of the measurement by the functional near-infrared spectroscopy.

5. The learning apparatus according to claim 1, wherein the information indicating the brain activity state of the subject is information that is based on characteristics of optical fibers at a time of the measurement by the functional near-infrared spectroscopy.

6. An estimation apparatus comprising:
an acquisition unit configured to acquire a functional image that is based on a measurement result obtained by measuring a brain activity state of a subject by functional near-infrared spectroscopy; and
an estimation unit configured to estimate a brain activity state of the subject based on the functional image acquired by the acquisition unit and a trained model that is generated in advance by machine learning with respect to a correlation between the functional image and brain activity information indicating states of Wernicke area and Broca area of the subject.
